(19) United States
(12) Patent Application Publication
(10) Pub. No.: US 2008/0027291 A1
(43) Pub. Date: Jan. 31, 2008

(54) Electronic patient compliance device

(76) Inventor: Wade E. Williams-Hartman, Wayne, NJ (US)

Correspondence Address: WARD & OLIVO, SUITE 300, 382 SPRINGFIELD AVENUE, SUMMIT, NJ, 07901, US

(21) Appl. No.: 11/827,461

(22) Filed: Jul. 11, 2007

Related U.S. Application Data
(60) Provisional application No. 60/830,049, filed on Jul. 11, 2006.

(51) Int. Cl. A61B 5/00

(52) U.S. Cl. 600/300

(57) ABSTRACT

An apparatus for monitoring patient compliance in the administration of medication includes a recording device for recording data related to administration of said medication by a patient, a collecting device and an electronic transmitting device for transmitting, via an electronic link, data to the collecting device. The data is related to administration of the medication and includes time and date of administration. The data may consist of many types of data such as patient identification information, medication identification information, dose consumed, reported side effects, severity of reported side effects, delays in consumption of said medication, text notes by said patient, and text notes by any other individual.

### CROSS-REFERENCE TO RELATED APPLICATIONS

This application claims the benefit of application Ser. No. 60/830,049, filed Jul. 11, 2006, which is incorporated herewith.

### FIELD OF THE INVENTION

The present invention relates to the field of administering medicine. Specifically, the present invention relates to monitoring patient compliance with a prescribed treatment regarding the time of consumption, frequency and amount of medicine taken.

### BACKGROUND OF THE INVENTION

Patient compliance is critical in the effective use of pharmaceuticals. The term “patient compliance” within the pharmaceutical industry refers to taking the proper type and amount of a medication at the proper time interval.

When new drugs are tested, compliance is extremely important in determining the effectiveness and side effects of drugs being tested. If patients skip doses, an effective drug may be mistakenly determined to be ineffective because it was not taken properly. Thus, millions of dollars of developmental costs as well as FDA approval may be lost by an otherwise effective drug due to lack of patient compliance. Conversely, negative side effects may not become evident during testing if patients do not take medication regularly enough to produce the negative side effect. The result could be that a potentially dangerous drug will be approved for use by the public. Drug recalls, negative publicity and lawsuits are all potential detriments which could be suffered by the companies involved in marketing a drug, as a result of lack of patient compliance.

Patient compliance is an important factor in determining effectiveness and side effects of new drugs, it also critical in the determination of proper dosage of a new drug. When patients don't take the prescribed amount of medication at the proper time, a new drug could be released into the market with recommendations for an improper dose. This may lead to lack of effectiveness, greater occurrence of side effects, and increased drug interactions.

While most interested parties would agree that compliance is extremely important, there are not many widely used systems for insuring patient compliance. Pre-printed fold-over blister cards are common in the clinical trial industry. Cards are printed with easy to understand graphics and text instructing the patient exactly what medication to take at the designated time. Slowly, such pre-printed cards are making their way into the commercial/retail marketplace with great success.

Easy to read instructions are a critical first step, yet they do little to provide actual patient compliance information back to the physician, clinician, or other involved party. Despite the fact that cards may have an area to write-in information about the time of dosing, patients often forget or may write false information in order to avoid criticism from the health care provider. It is possible that many individuals will skew information to show greater compliance as a means to achieve acceptance and avoid disapproval as well as possible termination as a participant in a clinical trial.

While there are some materials which pertain to the field of medication disbursement and recording information, such devices generally involve elaborate systems for dispensing the medication. Existing products attempt to control the dispensing of medication. Such systems are too complex to be cost effective, are too limiting with regard to the type of medications dispensed, may require repackaging of product into their dispensing system, tend to provide unreliable data, may not be child resistant, and can be perceived as too controlling by the patient.

U.S. Pat. No. 6,529,446 B1 to de la Huerga provides a good example of an elaborate system for dispensing medication. Exact time of dispensing is recorded via doors that open in the top of a system. While such a system has the potential of working, it is not portable, and is too large, awkward and expensive to be cost effective and convenient. Furthermore, any system such as this that controls or overtly monitors medication by opening doors or compartments can be perceived as overly controlling.

Attempts to include circuitry into blister cards can have two major drawbacks. First the cost of such a system is high. Each card may need to be manufactured with radio frequency ID chips, batteries or extensive circuitry wiring. Such high tech systems are delicate and may malfunction in normal conditions. Bending such a card could result in the computer recording doses taken when only a wire has been broken inside the card. False data will be an inevitability involved in such a systems and quickly invalidate patient data from a clinical trial. Thus, there is a need for a device which can monitor patients' adherence to medication regimens and is inexpensive. There is also a need for a device that is far less complex than other devices currently available.

### SUMMARY OF THE INVENTION

The present invention is an apparatus for monitoring patient compliance in the administration of medication. The apparatus includes a recording device for recording data related to administration of said medication to a patient, a collecting device and an electronic transmitting device for transmitting, via an electronic link, data to the collecting device. The data is related to administration of the medication and includes time and date of administration. The data may consist of many types of data such as patient identification information, medication identification information, dose consumed, reported side effects, severity of reported side effects, delays in consumption of said medication, text notes by said patient, and text notes by any other individual.

The medication and the apparatus are attached to a blister card and the device alerts a patient to the time of the next dosage of the medication.

Other objects, features and characteristics of the present invention, as well as the methods of operation and functions of the related elements of the structure, and the combination of parts and economies of manufacture, will become more apparent upon consideration of the following detailed description with reference to the accompanying drawings, all of which form a part of this specification.

### BRIEF DESCRIPTION OF THE FIGURES

A further understanding of the present invention can be obtained by reference to a preferred embodiment set forth in the illustrations of the accompanying drawings. Although the illustrated embodiment is merely exemplary of systems for carrying out the present invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

FIG. 1 is a diagram depicting a blister card embodiment of the present invention.

FIG. 2 is a diagram depicting a reminder device in accordance with an embodiment of the present invention.

FIG. 3 is a diagram depicting the transfer of information from the compliance monitoring device in accordance with an embodiment of the present invention.

FIG. 4 is a diagram depicting the changing of battery and reuse of the compliance monitoring device in accordance with an embodiment of the present invention.

FIGS. 5A & 5B are diagrams depicting a patient compliance monitoring system using a portable monitoring device in accordance with an embodiment of the present invention.

FIG. 6 is a diagram depicting the identification method to determine which medicine is taken in accordance with an embodiment of the present invention.

FIG. 7 is a flow chart depicting the steps taken when a patient enters side effect information in accordance with an embodiment of the present invention.

FIG. 8 is a flow chart depicting the steps taken when a patient delays the taking of a dose of medication in accordance with an embodiment of the present invention.

FIGS. 9 & 10 are diagrams depicting a patient compliance monitoring system meant to accommodate complex drug regimens in accordance with an embodiment of the present invention.

### DETAILED DESCRIPTION OF THE PREFERRED EMBODIMENTS

A detailed illustrative embodiment of the present invention is disclosed herein. However, techniques, systems and operating structures in accordance with the present invention may be embodied in a wide variety of forms and modes, some of which may be quite different from those in the disclosed embodiment. Consequently, the specific functional details disclosed herein are merely representative, yet in that regard, they are deemed to afford the best embodiment for purposes of disclosure and to provide a basis for the claims herein which define the scope of the present invention.

Moreover, well known methods and procedures for both carrying out the objectives of the present invention and illustrating the preferred embodiment are incorporated herein but have not been described in detail as not to unnecessarily obscure novel aspects of the present invention.

The present invention is a device for recording data (i.e. time, date and type of medication taken) related to patient compliance with drug regiments. Under clinical testing condition or when medication is administered to a patient in a hospital, the container in which the medicine is administered would contain an electronic device capable of recording the date and time when the device is activated, as well as any other relevant information, and transmitting such data to a central computer (i.e. server).

When a patient is given a drug the patient would activate the data recording device when taking the drug, by pressing a button on the device, the device would then record the time and date at which the patient consumed the medication and transmit this data to a data storing server. Such data can be reviewed by any relevant medical professional (i.e. doctor, testing technician, etc.) from a computer to ensure that a patient is compliant with medication regimens designed to either treat a patient or test a medication.

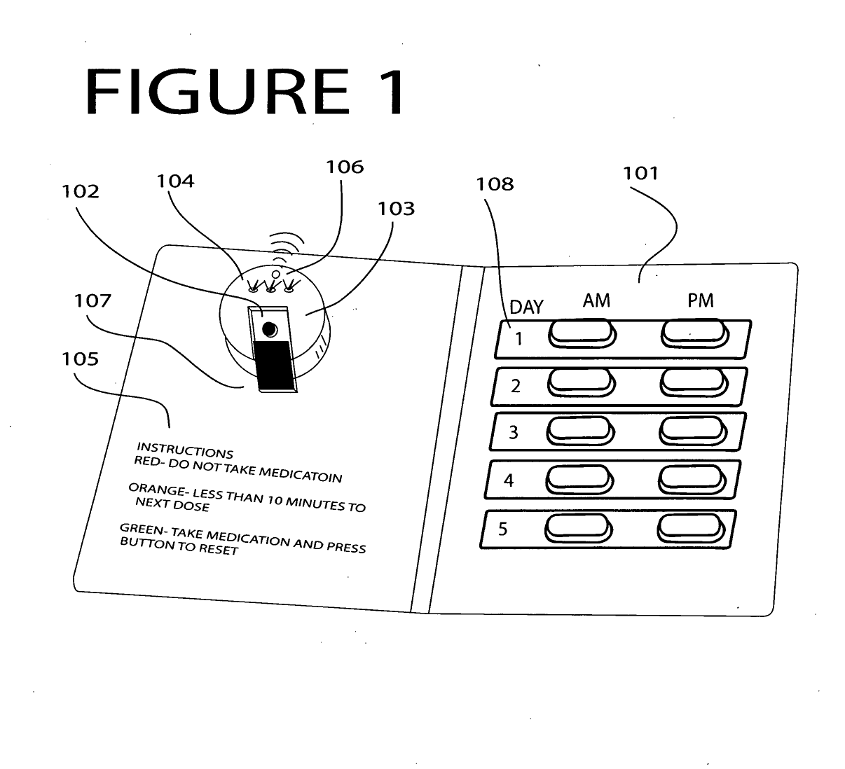

Referring to FIG. 1, the device of the present invention is shown. The device includes a blister card 101 a time and date recording device 103 which is attached to blister card 101. A simple button 102 for recording events is attached to device 103. The device 103 includes an alarm or reminder signal, such as a noise emitted by speaker 106 or light emitted by LED 104 that would indicate that the next dose of medication is due or overdue. Button 102 is used for event recording and has a protective cover 107 to prevent accidental event recording. Thus, the patient would have to slide or flip the protective cover 107 to expose the button 102 and then depress the button 102 to record that they have taken their medication. The blister card 101 would have clearly printed instructions 105 for the use of the time and date recording device 103 as well as the dosing instructions 108 for the medicine.

Rather than writing the time of dispensing the medication on the blister card 101, the patient will simply press the button 102 to record this event. Because the patient has no ability to alter the time/date on the device 103, they can not record information other than the actual time and date of each event.

Compliance with a schedule can be further increased with the addition of a reminder signal. The lighting of LED 104 with the speaker 106 emitting an audible alarm would remind the patient that it is time to take the medication. For example, a LED 104 that glows green 10 minutes before the exact prescribed time of dosing, indicating that it is now OK to take the medication, this could be combined with three audible beeps to attract attention.

If the patient has not already pressed the button 102 to record the event at the time of the dose, the speaker 106 would beep 5 times. The LED 104 would then turn to blinking orange 15 minutes after the recommended time of dosing. After a certain time period, when the dose should be skipped, the LED 104 would blink red. Printed instructions 105 on the card would direct the patient to respond to different colors of LED 104 and different audible signals emitted by speaker 106. One example would be that the patient would press the button 102 to reset the device and skip the dose and leave the dose in the card 101. Each medication or clinical trial could have its own customized design as to the precise compliance requirements. The signaling methods could be adjusted to cater to the needs of a particular study, without departing from the spirit of the present invention.

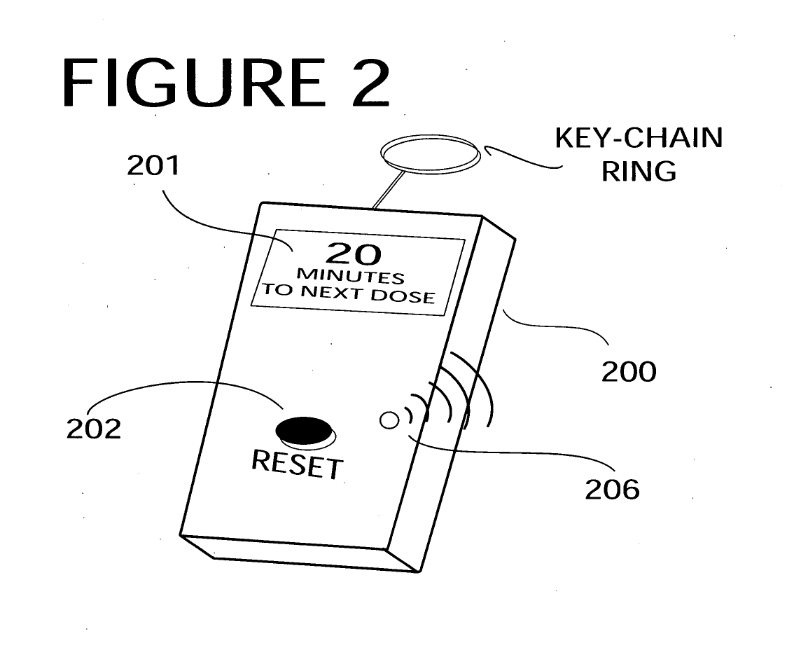

Shown in FIG. 2 is a reminder device 200. The reminder device 200 alerts the patient that it is time to take the medication. In this embodiment of the invention, the time and date recording device 103 is separate from the reminder device 200.

The time and date recording device 103 is adhered to the blister card as in FIG. 1, while the reminder device 200 is separate and portable as in FIG. 2. The portable component, namely reminder device 200, could provide the same type of audible reminder through a speaker 206 as the blister card 101 shown in FIG. 1.

Instead of the LED 104, a liquid crystal screen 201 could be used to display the exact amount of time until the next dose, or the amount of time until the dose is overdue. Only the device 103 adhered to the card would have the time/date recording button 102. This would reduce the ability of the patient to record an event unless they have the blister card 101 with the medication in their hand. The two devices synchronize with each other by means of radio frequency communication, or the reminder device 200 could simply have an alarm reset button 202 which would not record any actual time/date information.

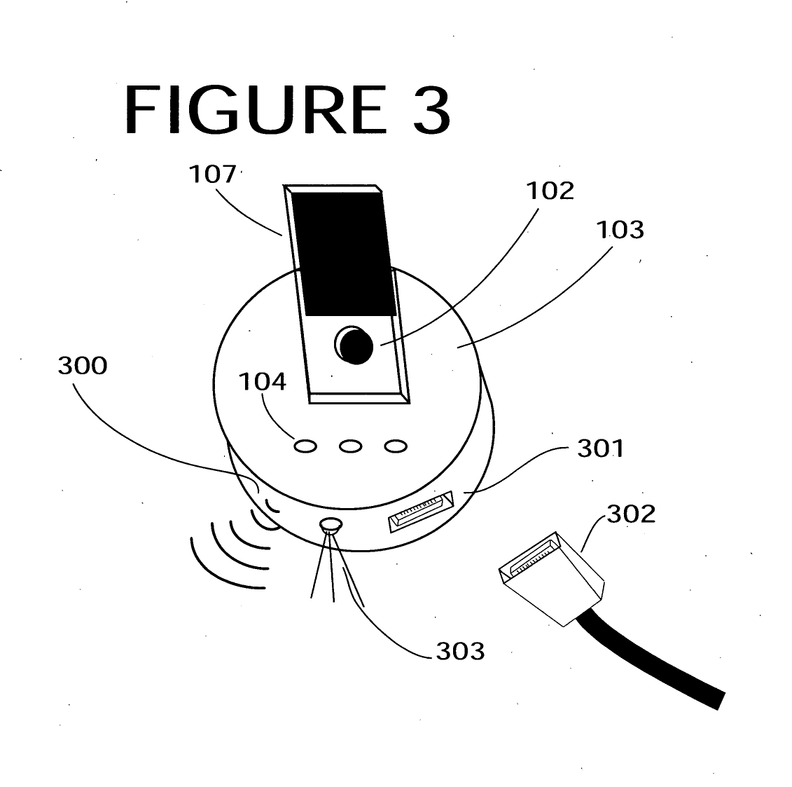

FIG. 3 shows the transfer of information from the compliance monitoring device. Once the blister card 101 has been used by the patient, it would be returned to the appropriate health care provider for analysis. As discussed with respect to FIG. 1, the recording device 103 includes a time and date recording button 102, a protective cover 107, LED 104. The device also includes a RF communication means 300, optical signal transmission means 303, and a wire socket 301.

The device 103 has a simple wire socket 301 to hook up wiring to the device 103. The patient downloads the information on device 103 to a computer for analysis by an electronic means such as radio frequency communication means 301, optical signal transmission means 303 (i.e. infrared LED), or by wire connection 302 to wire socket 301. Because the device 103 is not hidden inside the blister card 101, the data can be recovered by any conventional means. Other devices must rely on RFID transmission because they have been designed with no means to connect wires. Any other conceivable method of data transfer may be used without departing from the spirit of the present invention.

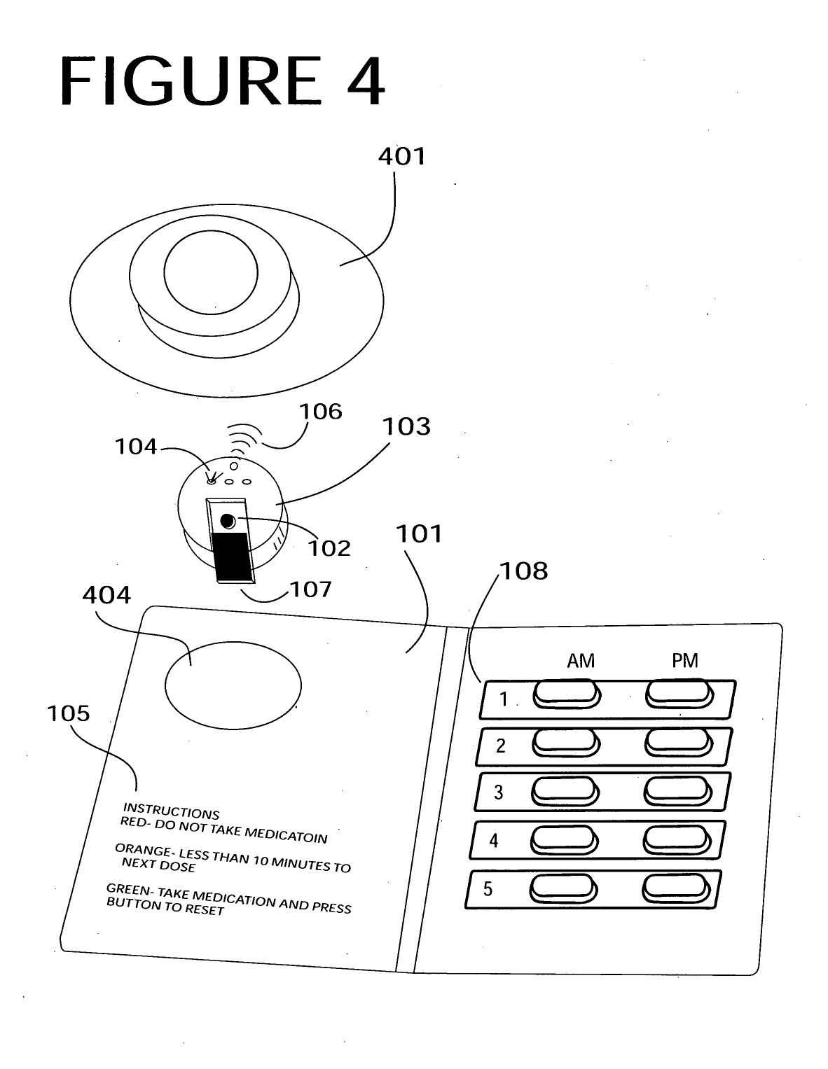

FIG. 4 shows the changing of battery and reuse of the compliance monitoring device. The electronic device 103 includes: a time and date recording button 102, a protective cover 107, LED indicators 104 and an audible alarm 106. Device 103 is designed to adhere to the card 101 during the time when the patient is in possession of the card 101. Once the data is recovered from the device 103, it is possible for the device 103 to be removed from the card 101, have a battery replaced and be reused on another card in the future. A flanged thermoformed blister 401 is used to adhere the electronic device 103 to the blister card 101, which contains dosage instructions 108. A die cut hole 404 in the top half of the blister card 101 will allow the flanged thermoformed blister 401 to be inserted at the time of sealing of the card 101. The ability to reuse the device 103 through several different cards will aid in cost reduction when utilizing the present invention. Even a device which is more expensive to manufacture becomes less costly when it is used multiple times over its life cycle.

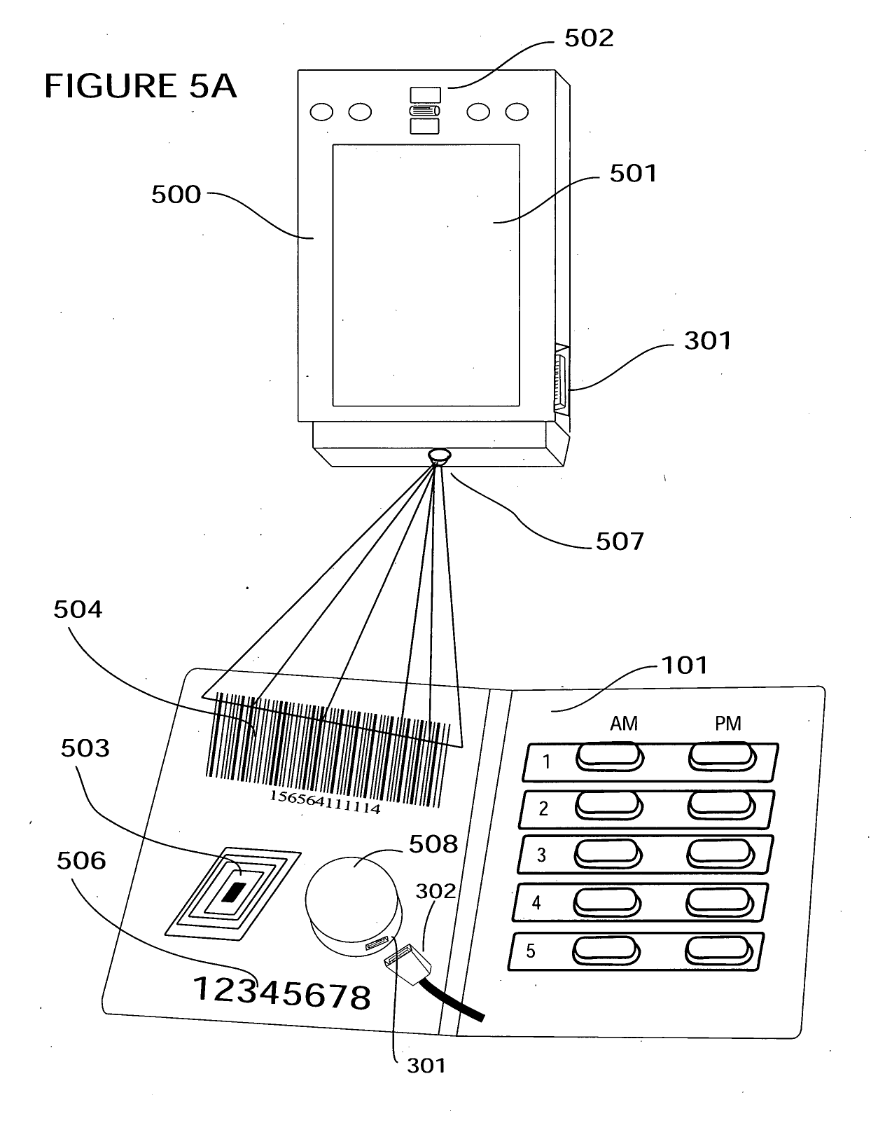

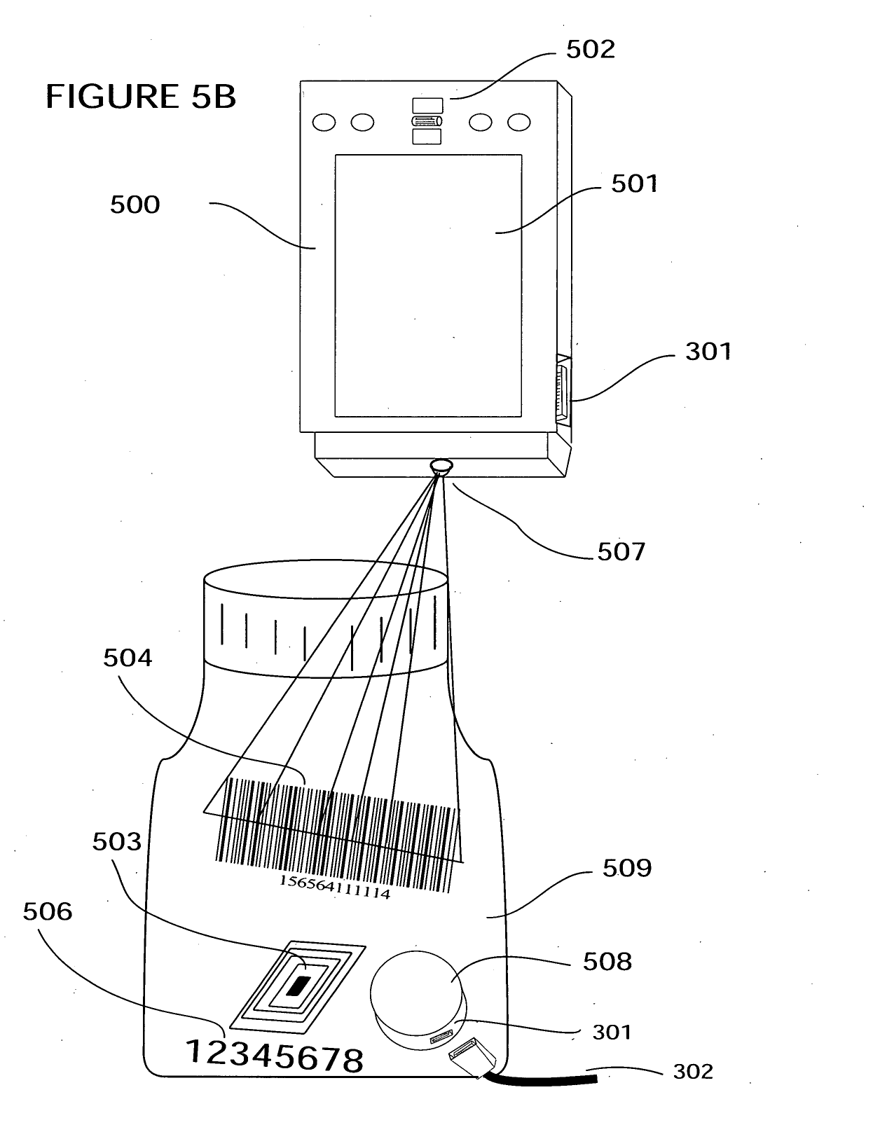

In yet another embodiment of the invention, as depicted in FIGS. 5A & 5B, the present invention uses more elaborate means to instruct the patient, and has a broader range of possible uses. In this configuration, more sophisticated electronic devices will be utilized. As shown in FIGS. 5A & 5B, the recording device is a small computerized device 500, with a screen 501 to display information, and data input means 502, such as a scroll wheel and buttons. Screen 501 can also be a touch screen for convenient data input.

Any other data input means maybe used without departing from the spirit of the present invention. It is possible that the computerized device 500 could be existing technology such an existing Personal Digital Assistant (PDA).

This device 500 could be configured to record multiple medication regimens. In such a scenario, it is preferred that the device 500 not be adhered to the container, whether it is a blister package 101 or a pill bottle 509. A method for reliably recording that the medication was present at the time of the event recording may be used. This could be established by the use of RFID tags 503 on or in each medicine container, the printing of barcodes 504 on the container, such as blister card 101 or pill bottle 509, wire socket 301, or the use of an alphanumeric code system 506.

With the RFID system, the card 101 containing the RFID tags 503 would need to be within the reading range of the computerized device 500, for the device 500 to acknowledge the medication and to record the event of the dispensing of that medication. With printed barcodes 504, the computerized device 500 would be equipped with a LED barcode reader 507 or a camera system to read the barcode 504. Wire hookup would entail a simple chip 508 possessing a socket 301 attached to the pill bottle 509 or blister package 101, and the data recording device to have a socket 301 capable of receiving the same type of wire connection 302 as the chip 508, thus verifying the presence and type of medication as well as identifying the medication.

In an alphanumeric system, the container would have information on it such as alpha numeric code 506 or the prescription number. The user would key in the number from the package in order to verify that they are indeed looking at the package. In this scenario, patients would be instructed not to copy down the alphanumeric code 506 elsewhere.

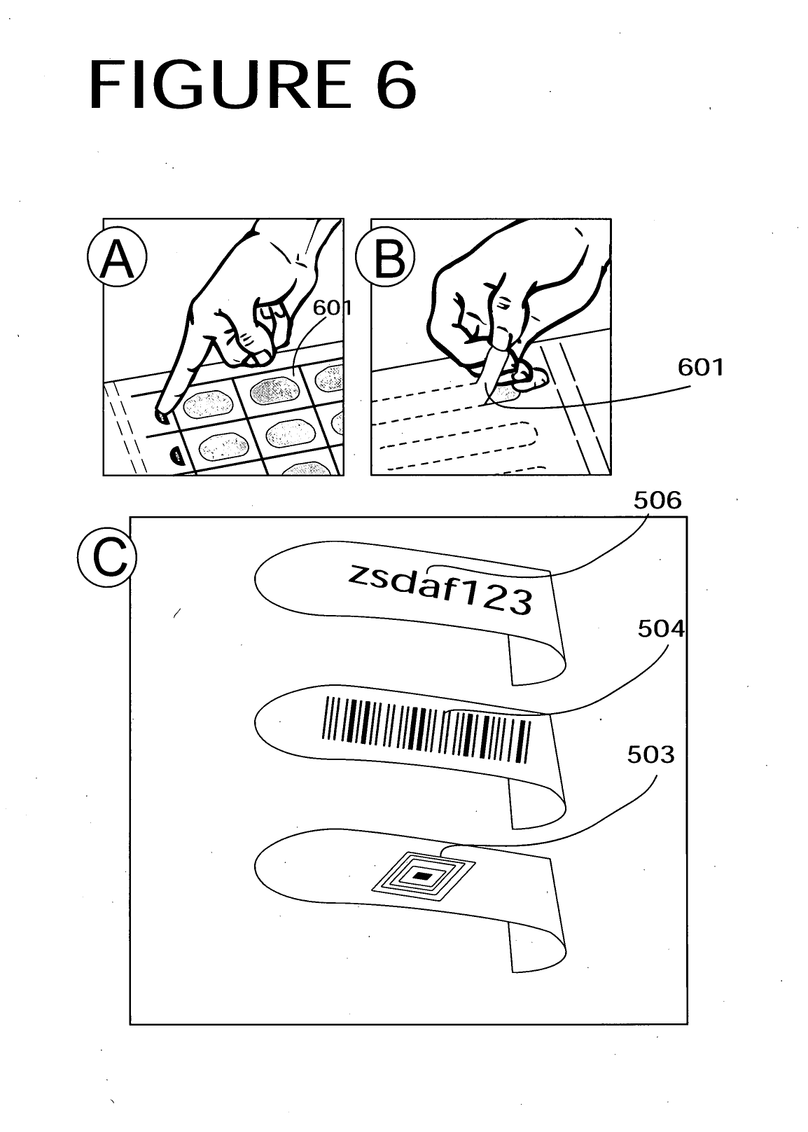

As shown in FIG. 6, the identification of which particular pill taken would be recorded when used with a blister card 101 or a similar container. In operation, a tab 601 would be removed from the back of the individual dose at the time of dispensing as shown in steps A & B in FIG. 6. This tab 601 could have a unique RFID tag 503, barcode 504, or alphanumeric code 506 on it. The user would record the identification of the pill which is taken in the same manner as in FIGS. 5A and 5B (i.e. RFID scanning, barcode scanning, etc.). Thus, when the dispensing event is recorded, the exact location on the blister card 101 of the medication is also recorded. Therefore, there would be a unique identifier for each dose.

The portable electronic data recording device 500 will allow more information to be recorded by the patient, and thus increase the amount of information to the healthcare provider. Because of the interactive nature of a PDA or similar device, the patient could be asked questions about their health at regular intervals, such as at the time of dosing of medication. Through software programming, the questions could be adapted to the responses of the patient.

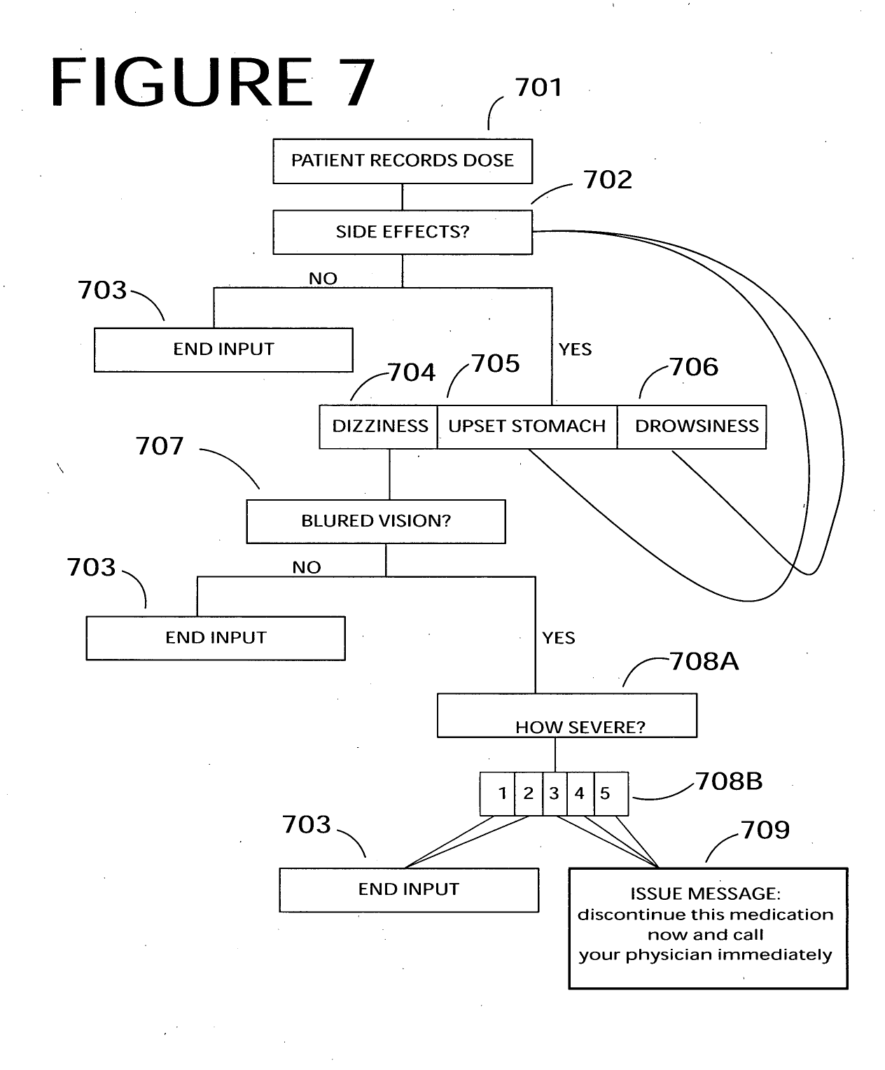

FIG. 7 shows a flow chart of the steps taken when a patient enters side effect information. These reports could be followed up with further questions in order to get more detailed information about the negative event. For example, the patient first records the dose taken in step 701. The system asks if there are any negative effects in step 702. If the patient responds no, the input process would end in step 703. However, if the patient's response is yes in step 702 the system would prompt for the type of side effect. The patient would choose from a list of side effects such as: dizziness 704, upset stomach 705, and drowsiness 706. Then in step 707, the system would ask a follow up question related to a described side effect, such as “is the participant experiencing blurred vision?” If the patient answers “no”, the process ends in step 703. If the patient answers “yes” to step 707, the patient would be further asked to describe the severity of the symptoms in step 708A on a scale of 1-5 708B. The system would further issue patient instructions such as “discontinue this medication now and call your physician immediately” in step 709 if necessary, or if not end the inquiry process in step 703.

By asking the questions at time of the event, the patient can provide more accurate responses to the questions than if they were to wait until their next doctor's appointment. Furthermore, when necessary, the system would follow-up with more in-depth questions thus providing more feedback to the physician. By having the system ask a number of predetermined questions closer to the event, important questions will be asked. When the information is downloaded to the healthcare provider, problems could be flagged for follow up, thus reducing the possibility that a serious side effect or problem will be overlooked. Recording of the exact time line of positive effects of a drug can be critical information for the physician or clinician.

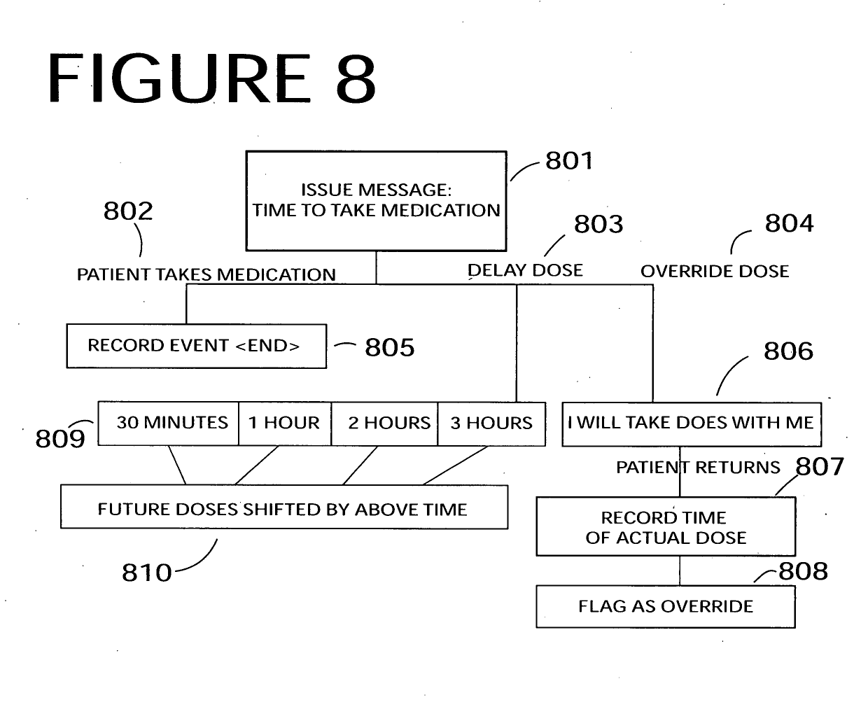

FIG. 8 shows a flow chart depicting the steps taken when a patient delays the taking of a dose of medication. The system aids the patient in establishing a dosing regimen that works for their individual lifestyle. For example, a message would issue informing the user that it is time to take a medication in step 801. The user may then either: take the medication in step 802, delay the dose in step 803, or override the dose in step 804. If the patient takes the dose at the prescribed time the adjustment process ends in step 805. If the patient chooses to delay the dose, a time frame of delay is chosen in step 809, and the time of future doses is shifted in step 810 by the appropriate time frame.

If the patient overrides the dose in step 804 the patient may choose to inform the system that the medication will be taken later in step 806. This may be done outside of observation. Once the patient returns to the testing facility or observation area the time when the patient ingested the medication is recorded in step 807. Then that particular dose is flagged as an overridden dose in step 808.

Patients with too many dose overrides could be identified and reviewed. In the case of a clinical trial, non-compliant participant's data could be reviewed and/or removed from the drug study. In the general population, physicians would be alerted to the lack of patient compliance as part of their overall assessment of the patient's condition.

This system can also be used to instruct patients how to handle missed or skipped doses. Because the system knows how long it has been since the last dose, it could instruct the patient to take double the medication, skip a dose, or set up an accelerated dosing pattern to get the patient back on the proper schedule. The exact choice for such handing of missed doses would be pre-programmed into the system by the health care provider and can be tailored to the particular medication taken.

It is also contemplated that drug interaction of existing medications could be uploaded into the system, and that all medications would be checked against each other to reduce the possibility of interaction.

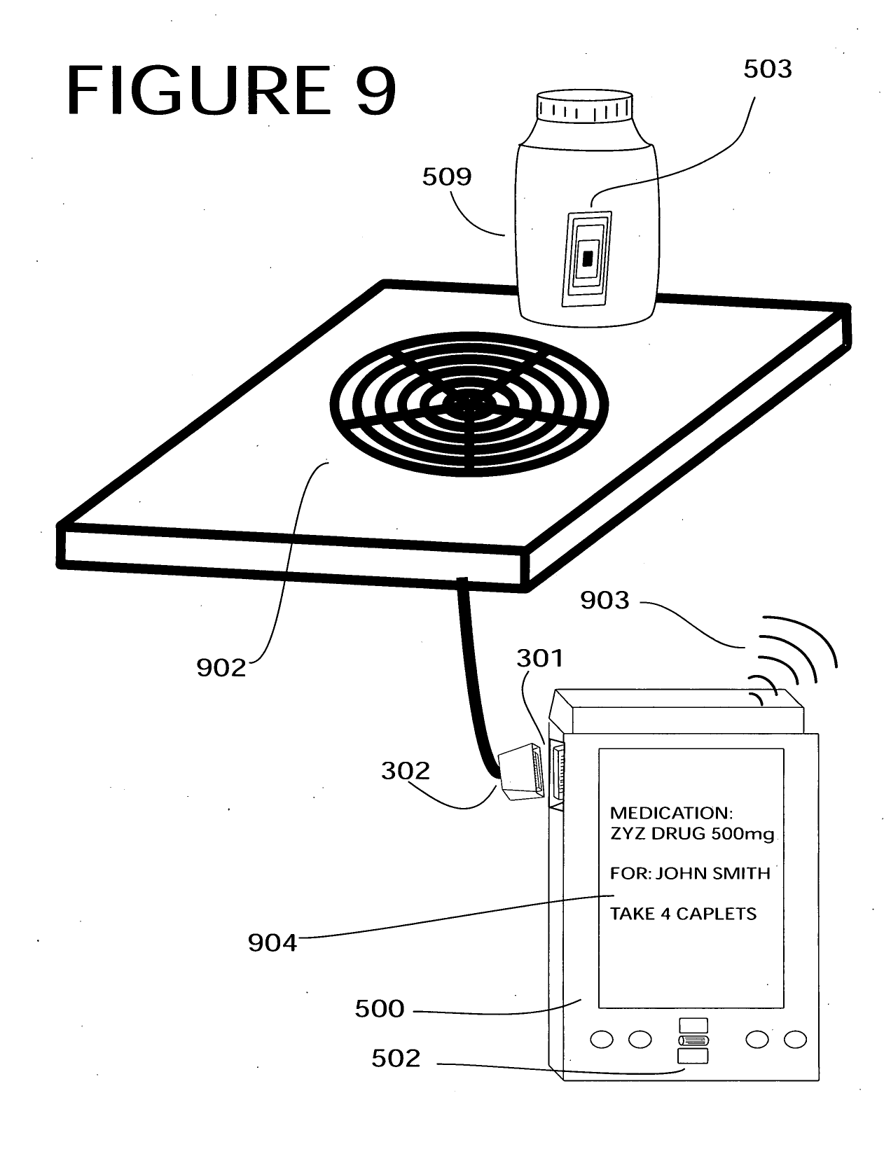

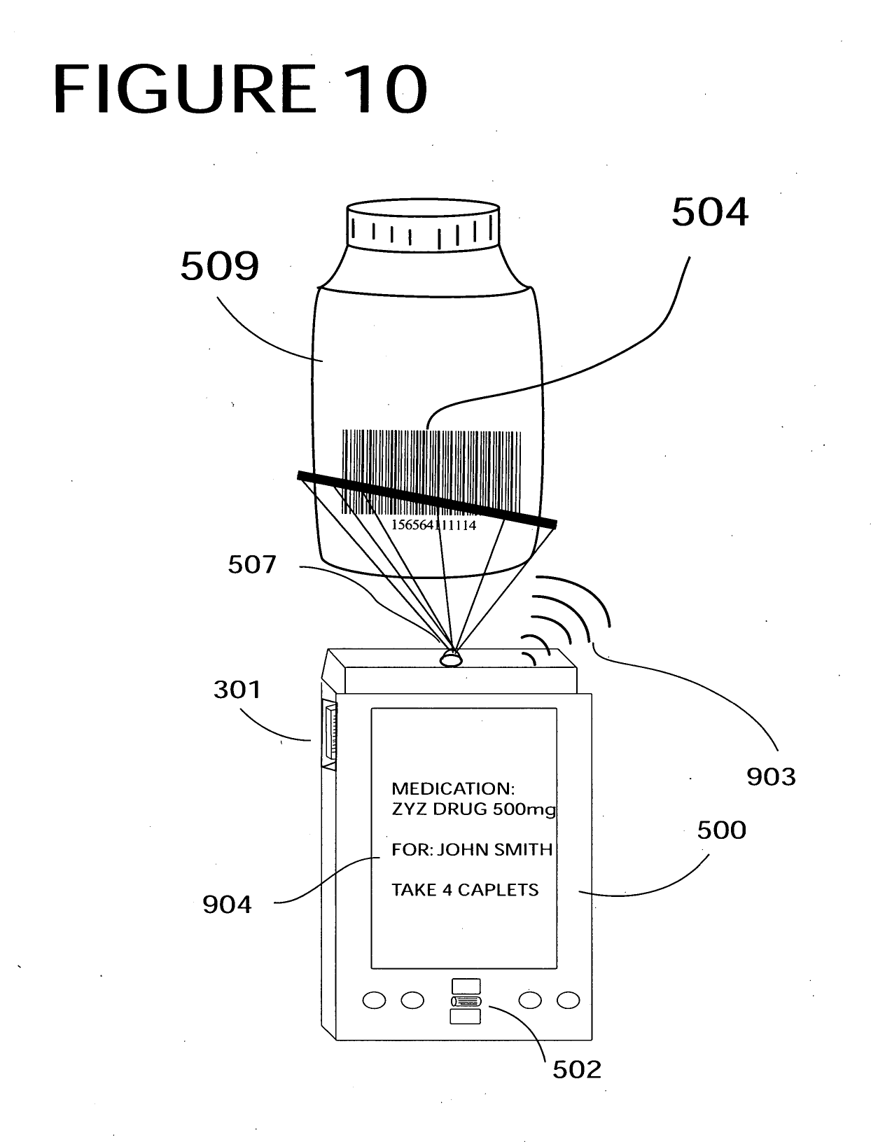

Yet another embodiment of the device is depicted in FIGS. 9 and 10. This embodiment is especially useful in assisting individuals who are: elderly, visually impaired, mentally impaired, or individuals who are on complex drug regimens. This embodiment would incorporate elaborate dosing instructions for the patient.

The system shown in FIG. 9 includes a medicine bottle 509 possessing a RFID tag 503, a RFID reader mat 902 possessing a wire connection 302, a computerized device 500 possessing a wire socket 301 capable of coupling with wire connection 302 and displaying instructions 904 relevant to the medication in bottle 509. The pharmacist would dispense medication in a bottle 509 with a RFID tag 503 on it. The system would prompt the patient at the time of dosing. The patient would place the medicine containers on a RFID reader mat 902, the system would audibly speak the name of the prescription using speaker 903, indicating the number of pills to be taken at that time. Instructions 904 confirming amount of medicine in each dose would be large and easy to read.

The system in FIG. 10 includes a medicine bottle 509 possessing a barcode 504, computerized device 500 possessing a wire socket 301 capable of coupling with wire connection 302 and displaying instructions 904 relevant to the medication in bottle 509, the pharmacist would dispense medication in a bottle 509 including a barcode 504. The system would prompt the patient at the time of dosing. The patient would scan the barcode using barcode scanner 507, the system would then audibly speak the name of the prescription using speaker 903, indicating the number of pills to be taken at that time. Instructions 904 confirming amount of medicine in each dose would be large and easy to read.

Confirmation of dosing can be received via voice recognition. The system would also be able to warn patients of mixed up medicine containers between individuals in a household. Thus a wife would be less likely to take a husband's medication because all medication would be identified by system prior to dispensing.

Additionally, the electronic device which monitors compliance can be read by the doctor at regular check-up intervals. If the device was hooked up to a central computer at the user's home or at the pharmacy, non-compliance could be identified and automatic emails to the physician and/or responsible caregivers (i.e. family) would be generated to alert them that the patient has exceeded some minimum compliance requirement. An example would be that if a patient misses 3 or more doses in a week and their caretaker and the physician are alerted via email, text message or other electronic means. By downloading the compliance data to a home PC or other device with internet access, such non-compliance would be detected quickly, before health consequences occur.

Up to the minute recording of medication is critical to emergency care givers responding to patients in medical crisis. Information about the previous 48 hours of medication is of utmost importance in such cases. In such a scenario, the EMT would simply have to bring the medication computer to the emergency room to read a full record of the patient's current medications, dosing levels and time of dosing.

Elaborate dosing regimens such as AIDS drugs and diabetes medication would be excellent uses of the present invention. It is contemplated that the system could easily interface with blood glucose readers to record critical blood sugar levels as well as to record the medication taken. This could include user input data for injected insulin as well as recoding information about current health conditions.

By recording this data in one device, data analysis would be greatly simplified, with trends and statistical factors more accurately identified. Even though complex regimens would be most important for this system, it could be used by anyone on a drug therapy to help boost compliance and aid in the convenience of medication dispensing.

Once data is downloaded, it can be analyzed by software to determine the compliance rate of a given patient. Coupled with medical records, compliance can be factored into the effectiveness rating of the drug. With this critical information, bad data may be eliminated from drug studies leading to more accurate evaluations of drugs under review.

In the case of the consumer, the physician may encourage stronger adherence to the dosing instructions before changing a medication. The result could be greater effectiveness of preferred medications; thus reducing the reliance on more dangerous medications, surgeries, or other intrusive therapies. In an environment where information is critical to accurate decision making, accurate dosing information and compliance to the prescribed regimen is critical. A simple, low cost, easy to use device can greatly aid in the flow of information back to the health care provider.

While the present invention has been described with reference to the preferred embodiment and alternative embodiments, which have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

What is claimed:

1. A method for monitoring patient compliance in administration of medication, the method comprising the steps of:
administering the medication to a patient;
recording data related to the administering of the medication;
transmitting, via an electronic means the data to a collection device; and
maintaining records of the recorded data.

2. The method of claim 1 wherein the data related to consumption of the medication includes time and date of administration.

3. The method of claim 2 wherein the data related to administering of the medication is chosen from a group consisting of: patient identification information, medication identification information, dose consumed, reported side effects, severity of reported side effects, delays in consumption of said medication, text notes by said patient, and text notes by any other individual.

4. The method of claim 1 wherein the medication is contained in a blister card.

5. The method of claim 4 wherein the recording data is accomplished by an electronic device and a data input means, the data input means being attached to the blister card.

6. The method of claim 5 wherein the device is removably attached to the blister card.

7. The method of claim 1 wherein the electronic means comprises a wire.

8. The method of claim 1 wherein the electronic means comprises an infrared transmitter.

9. The method of claim 1 wherein the electronic means comprises a RF transmitter.

10. The method of claim 1 wherein the electronic means comprises a wireless transmitter.

11. The method of claim 1 wherein the recording of data is accomplished by an autonomous intelligent device possessing a data input means.

12. The method of claim 1 wherein the step of recording data related to the administration of the medication further comprises the step of scanning a barcode from a medication container, wherein the barcode contains information related to the medication.

13. The method of claim 1 wherein the step of recording data related to consumption of the medication further comprises the step of scanning a RF identifier from a medication container, wherein the RF identifier contains information related to the medication.

14. The method of claim 1 wherein the collecting device is an electronic system capable of maintaining, storing, receiving and allowing access to data.

15. An apparatus for monitoring patient compliance in administration of medication, the apparatus comprising:
recording device for recording data related to administration of said medication by a patient;
collecting device; and
an electronic transmitting device for transmitting via an electronic link said data to said collecting device.

16. The apparatus of claim 15 wherein said data related to administration of said medication includes: time and date of administration.

17. The apparatus of claim 16 wherein said data related to consumption of said medication is chosen from a group consisting of: patient identification information, medication identification information, dose consumed, reported side effects, severity of reported side effects, delays in consumption of said medication, text notes by said patient, and text notes by any other individual.

18. The apparatus of claim 15 wherein said medication is contained in a blister card.

19. The apparatus of claim 18 wherein said apparatus is attached to said blister card.

20. The apparatus of claim 19 wherein said apparatus is removably attached to said blister card.

21. The apparatus of claim 15 wherein said apparatus alerts said patient to the time of the next dosage of said medication.

22. The apparatus of claim 15 wherein said electronic link comprises a wire.

23. The apparatus of claim 15 wherein said electronic link comprises an infrared link.

24. The apparatus of claim 15 wherein said electronic link comprises a RF link.

25. The apparatus of claim 15 wherein said electronic link comprises a wireless link.

26. The apparatus of claim 15 wherein said apparatus is an autonomous intelligent device.

27. The apparatus of claim 26 wherein said autonomous intelligent device includes a display means.

28. The apparatus of claim 27 wherein said display means is touch sensitive.

29. The apparatus of claim 27 wherein said autonomous intelligent device includes an optical scanner.

30. The apparatus of claim 27 wherein said autonomous intelligent device includes a camera.

31. The apparatus of claim 15 wherein said collecting device is an electronic system capable of maintaining, storing, receiving and allowing access to data.
